(19) United States
(12) Patent Application Publication
(10) Pub. No.: US 2012/0029568 A1
(43) Pub. Date: Feb. 2, 2012

(54) Spinal connecting members with radiused rigid sleeves and tensioned cords

(76) Inventors: Roger P. Jackson, Prairie Village, KS (US); James L. Surber, Kansas City, KS (US)

(21) Appl. No.: 13/136,331

(22) Filed: Jul. 28, 2011

Related U.S. Application Data
(63) Continuation-in-part of application No. 12/802,849, filed on Jun. 15, 2010.
Application No. 12/802,849 is a continuation-in-part of application No. 12/924,802, filed on Oct. 5, 2010.
Application No. 12/924,802 is a continuation-in-part of application No. 12/221,442, filed on Aug. 1, 2008.
Application No. 12/221,442 is a continuation-in-part of application No. 11/328,481, filed on Jan. 9, 2006, now Pat. No. 7,862,587.
Application No. 11/328,481 is a continuation-in-part of application No. 12/148,465, filed on Apr. 18, 2008.
Application No. 12/148,465 is a continuation-in-part of application No. 12/661,042, filed on Mar. 10, 2010.
(60) Provisional application No. 61/400,504, filed on Jul. 29, 2010.
(60) Provisional application No. 61/403,915, filed on Sep. 23, 2010.
(60) Provisional application No. 61/268,708, filed on Jun. 15, 2009.
(60) Provisional application No. 61/270,754, filed on Jul. 13, 2009.
(60) Provisional application No. 61/336,911, filed on Jan. 28, 2010.
(60) Provisional application No. 61/395,564, filed on May 14, 2010.
(60) Provisional application No. 61/395,752, filed on May 17, 2010.
(60) Provisional application No. 61/396,390, filed on May 26, 2010.
(60) Provisional application No. 60/927,111, filed on May 1, 2007.
(60) Provisional application No. 61/210,058, filed on Mar. 13, 2009.

(51) Int. Cl. A61B 17/70

(52) U.S. Cl. 606/264

(57) ABSTRACT

A spinal implant having at least two bone anchors includes a longitudinal connecting member assembly having hard, non-elastic sleeves for attachment to the bone anchors, at least one spacer engaging the bone anchors and the sleeves, and in some embodiments, an end elastic bumper. A cord is initially slidingly received within the rigid sleeves, the spacer and the bumper. The sleeves include a lower radiused surface for direct engagement with a radiused surface of a shank of the cooperating bone anchor. The sleeves include an aperture for receiving a closure top portion for locking the cord against the sleeve, or alternatively receiving a closure top that does not extend into the aperture, the slip or grip option provided by the aperture in each sleeve resulting in an overall connector with variable segmental stiffness.

### CROSS-REFERENCE TO RELATED APPLICATIONS

This application claims the benefit of U.S. Provisional Patent Application Ser. No. 61/400,504 filed Jul. 29, 2010 and U.S. Provisional Patent Application Ser. No. 61/403,915 filed Sep. 23, 2010, both of which are incorporated by reference herein.

This application is also a continuation-in-part of U.S. patent application Ser. No. 12/802,849 filed Jun. 15, 2010 that claims the benefit of the following U.S. Provisional Patent Application Serial Nos.: 61/268,708, filed Jun. 15, 2009; 61/270,754, filed Jul. 13, 2009; 61/336,911 filed Jan. 28, 2010; 61/395,564 filed May 14, 2010; 61/395,752 filed May 17, 2010; and 61/396,390 filed May 26, 2010; all of which are incorporated by reference herein. This application is also a continuation-in-part of U.S. patent application Ser. No. 12/924,802 filed Oct. 5, 2010 that is incorporated by reference hereon. This application is also a continuation-in-part of U.S. patent application Ser. No. 12/221,442 filed Aug. 1, 2008 that is a continuation-in-part of U.S. patent application Ser. No. 11/328,481 filed Jan. 9, 2006, both of which are also incorporated by reference herein. This application is also a continuation-in-part of U.S. patent application Ser. No. 12/148,465 filed Apr. 18, 2008 that claims the benefit of U.S. Provisional Patent Application Ser. No. 60/927,111 filed May 1, 2007, both of which are incorporated by reference herein. This application is also a continuation-in-part of U.S. patent application Ser. No. 12/661,042 filed Mar. 10, 2010 that claims the benefit of U.S. Provisional Patent Application Ser. No. 61/210,058 filed Mar. 13, 2009, both of which are incorporated by reference herein.

### BACKGROUND OF THE INVENTION

The present invention is directed to dynamic fixation assemblies for use in bone surgery, particularly spinal surgery, and in particular to longitudinal connecting members and cooperating bone anchors or fasteners for such assemblies, the connecting members being attached to at least two bone anchors.

Historically, it has been common to fuse adjacent vertebrae that are placed in fixed relation by the installation therealong of bone screws or other bone anchors and cooperating longitudinal connecting members or other elongate members. Fusion results in the permanent immobilization of one or more of the intervertebral joints. Because the anchoring of bone screws, hooks and other types of anchors directly to a vertebra can result in significant forces being placed on the vertebra, and such forces may ultimately result in the loosening of the bone screw or other anchor from the vertebra, fusion allows for the growth and development of a bone counterpart to the longitudinal connecting member that can maintain the spine in the desired position even if the implants ultimately fail or are removed. Because fusion has been a desired component of spinal stabilization procedures, longitudinal connecting members have been designed that are of a material, size and shape to largely resist bending (flexion, extension and lateral), torsion, shear, distraction and compression, and thus substantially immobilize the portion of the spine that is to be fused. Thus, longitudinal connecting members are typically uniform along an entire length thereof, and usually made from a single or integral piece of material having a uniform diameter or width of a size to provide substantially inelastic rigid support in all planes.

An alternative to fusion, which immobilizes at least a portion of the spine, and the use of more rigid longitudinal connecting members or other rigid structure has been a “soft” or “dynamic” stabilization approach in which a flexible loop-, S-, C- or U-shaped member or a coil-like and/or a spring-like member is utilized as an elastic longitudinal connecting member fixed between a pair of pedicle screws in an attempt to create, as much as possible, a normal loading pattern between the vertebrae in flexion, extension, side bending, distraction, compression and torsion. Another type of soft or dynamic system known in the art includes bone anchors connected by flexible cords or strands, typically made from a plastic material. Such a cord or strand may be threaded through cannulated spacers that are disposed between adjacent bone anchors when such a cord or strand is implanted, tensioned and attached to the bone anchors. The spacers typically span the distance between bone anchors, providing limits on the bending movement of the cord or strand and thus strengthening and supporting the overall system. Shear forces are not well resisted by the typical cord and spacer stabilization systems. Such tensioned cord and spacer systems may also cause facet joint compression during spinal movement, especially flexion.

The complex dynamic conditions associated with spinal movement create challenges for the design of elongate elastic longitudinal connecting members that exhibit an adequate fatigue strength to provide stabilization and protected motion of the spine, without fusion, and that allow for some natural movement of the portion of the spine being reinforced and supported by the elongate elastic or flexible connecting member. A further challenge are situations in which a portion or length of the spine requires a more rigid stabilization, possibly including fusion, while another portion or length may be better supported by a more dynamic system that allows for protective movement.

### SUMMARY OF THE INVENTION

Longitudinal connecting member assemblies according to the invention for use between at least two bone anchors provide dynamic, protected motion of the spine and may be extended to provide additional dynamic sections or more rigid support along an adjacent length of the spine, with fusion, if desired. A dynamic longitudinal connecting member assembly according to the invention has an inner segment or core made from a cord in the disclosed embodiment, the cord being tensioned and fixed at least at either end of the assembly. The cord is received by at least one hard, rigid, inelastic segment or sleeve, the sleeve attachable to at least one bone anchor. Sleeves of the invention include a surface for direct engagement with a shank of a polyaxial bone screw. Such a surface may be configured as being concave, with a radius the same or similar to an upper convex domed surface of the shank and/or a spherical or otherwise curved surface at or near an upper surface of the shank. In some embodiments, the cord is received by at least a pair of such sleeves, each sleeve attachable to a bone anchor. In some embodiments, the sleeve or sleeves slidingly receive the cord. In other embodiments, the sleeve or sleeves are either fixed or left unfixed to the cord by the surgeon, resulting in a connecting member having variable segmental stiffness along a length thereof. A variety of embodiments according to the invention are possible. Additional sleeves may be attached to additional bone anchors and cooperate with additional cut-to-length spacers to create longer assemblies. Sleeves may also be extended to provide inelastic rod, bar or tube extensions, especially on one end. Spacers with different measures of rigidity may be connected according to embodiments of the invention. Either rigid lengths or cords may be of greater or lesser lengths for attaching to one or a plurality of bone anchors. In some embodiments, longitudinal connecting member assemblies may be dynamically loaded before insertion, or after being operatively attached to at least a pair of bone anchors along a patient's spine by tensioning the inner cord and at least partially compressing an end bumper and/or at least one spacer located between the bone anchors. Typically, the at least one spacer has some flexibility in bending, with the spacer protecting and limiting flexing movement of the inner core and providing shear resistance.

An object of the invention is to provide lightweight, reduced volume, low profile assemblies for use with at least two bone anchors. Furthermore, it is an object of the invention to provide apparatus and methods that are easy to use and especially adapted for the intended use thereof and wherein the apparatus are comparatively inexpensive to make and suitable for use.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a perspective view of a set of longitudinal connecting member components according to the invention, in particular a first sleeve, a second sleeve and a sleeve/rod coupler.

FIG. 2 is an enlarged perspective view of the first sleeve of FIG. 1 shown assembled with a polyaxial bone screw assembly, in partial perspective view, cooperating with a slide or slipping closure top.

FIG. 2a is a partial perspective view, similar to

FIG. 2 showing a modified, flangeless sleeve assembled with the polyaxial bone screw assembly of FIG. 2.

FIG. 3 is a reduced and exploded front elevational view of the entire assembly of FIG. 2 and further shown with two closure tops: the slide or slipping closure top (on left) of FIG. 2 and an alternative cord gripping closure top (on right).

FIG. 4 is a reduced top plan view of the sleeve shown in FIG. 2.

FIG. 5 is a bottom plan view of the sleeve of FIG. 4.

FIG. 6 is a front elevational view of the sleeve of FIG. 4.

FIG. 7 is a side elevational view of the sleeve of FIG. 4.

FIG. 8 is an enlarged cross-sectional view taken along the line 8-8 of FIG. 4.

FIG. 9 is an enlarged and partial side elevational view of the assembly shown in FIG. 2 with portions broken away to show the detail thereof and shown with a cord (in phantom).

FIG. 10 is an enlarged and partial side elevational view of the second sleeve illustrated in FIG. 1 shown with the bone screw assembly of FIG. 3 in side elevation, and further assembled with the alternative cord gripping closure top of FIG. 3, also shown in side elevation.

FIG. 11 is a partial side elevational view of the assembly shown in FIG. 10 with portions broken away to show the detail thereof and further shown with a cord (in phantom).

FIG. 12 is a reduced perspective view of the sleeve/rod coupler of FIG. 1 shown with the bone screw assembly of FIG. 3 and the alternative cord gripping closure top of FIG. 3, also in reduced perspective view.

FIG. 13 is an enlarged and partial front elevational view of the assembly of FIG. 12.

FIG. 14 is an enlarged and partial side elevational view of the assembly of FIG. 12 with portions broken away to show the detail thereof.

FIG. 15 is a partial perspective view of a longitudinal connecting member assembly that includes sleeves of the invention, a cord, an end blocker assembly and compressible spacers and is shown with each sleeve attached to a bone screw of FIG. 3, shown in reduced and partial perspective view and partially exploded to show sliding or fixed cooperation of the cord with the closure tops shown in FIG. 3.

FIG. 16 is a perspective view of another set of longitudinal connecting member components according to the invention, in particular a first sleeve with parallel flanges, a second sleeve with parallel flanges and tubular extensions, a third sleeve with lordotic flanges and tubular extensions and a sleeve/rod coupler.

FIG. 17 is a partial perspective view of the first sleeve of FIG. 16 shown in a stage of assembly with a polyaxial bone screw having portions broken away to show the detail thereof.

FIG. 18 is a partial perspective view, with portions broken away, similar to FIG. 17, showing the polyaxial bone screw of FIG. 17 fully assembled with the first sleeve and the slide or slipping closure of FIGS. 2 and 3.

FIG. 19 is an enlarged and partial side elevational view of the assembly of FIG. 18 with portions broken away to show the detail thereof and showing the bone screw shank disposed at an angle with respect to the receiver thereof.

FIG. 20 is a reduced top plan view of the second sleeve shown in FIG. 16.

FIG. 21 is a bottom plan view of the sleeve of FIG. 20.

FIG. 22 is a side elevational view of the sleeve of FIG. 20.

FIG. 23 is a front elevational view of the sleeve of FIG. 20.

FIG. 24 is a cross-sectional view taken along the line 24-24 of FIG. 20.

FIG. 25 is an enlarged and partial perspective view of the assembly of FIG. 18, but including the second sleeve of FIG. 20 in lieu of the first sleeve shown in FIG. 18 and with portions broken away to show the detail thereof.

FIG. 26 is a partial perspective view of the assembly of FIG. 25, but including the third lordotic sleeve of FIG. 16 in lieu of the second sleeve shown in FIG. 25 and with portions broken away to show the detail thereof.

FIG. 27 is a reduced and partial side elevational view of the assembly of FIG. 26 with portions broken away to show the detail thereof.

FIG. 28 is an alternative perspective view of the sleeve/rod coupler of FIG. 16.

FIG. 29 is a side elevational view of the sleeve/rod coupler of FIG. 28 with portions broken away to show the detail thereof.

FIG. 30 is another perspective view of the sleeve/rod coupler of FIG. 28 shown assembled with the bone screw assembly of FIG. 18 in lieu of the sleeve shown in FIG. 18, the bone screw assembly shown in partial, perspective view and with portions broken away to show the detail thereof.

FIG. 31 is a partial perspective view of another longitudinal connecting member assembly that includes sleeves of the invention the same or similar to those shown in FIG. 16, a cord, an end blocker assembly and compressible spacers and is shown with each sleeve attached to a bone screw of FIG. 18, shown in reduced and partial perspective view and partially exploded to show sliding or fixed cooperation of the cord with the closure tops shown in FIG. 3.

FIG. 32 is partial front elevational view of an alternative bone screw shank for use with longitudinal connecting members and sleeves of the invention, the shank spherical head hemisphere being shown in phantom.

FIG. 33 is a perspective view of the shank of FIG. 32.

FIG. 34 is an enlarged and partial side elevational view of the assembly of FIG. 18 with portions broken away, similar to FIG. 19 with the exception that the bone screw shank shown in FIG. 19 has been replaced with the bone screw shank of FIGS. 32 and 33, also with portions broken away to show the detail thereof.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of the connecting member assemblies of the application and cooperating bone anchors in actual use.

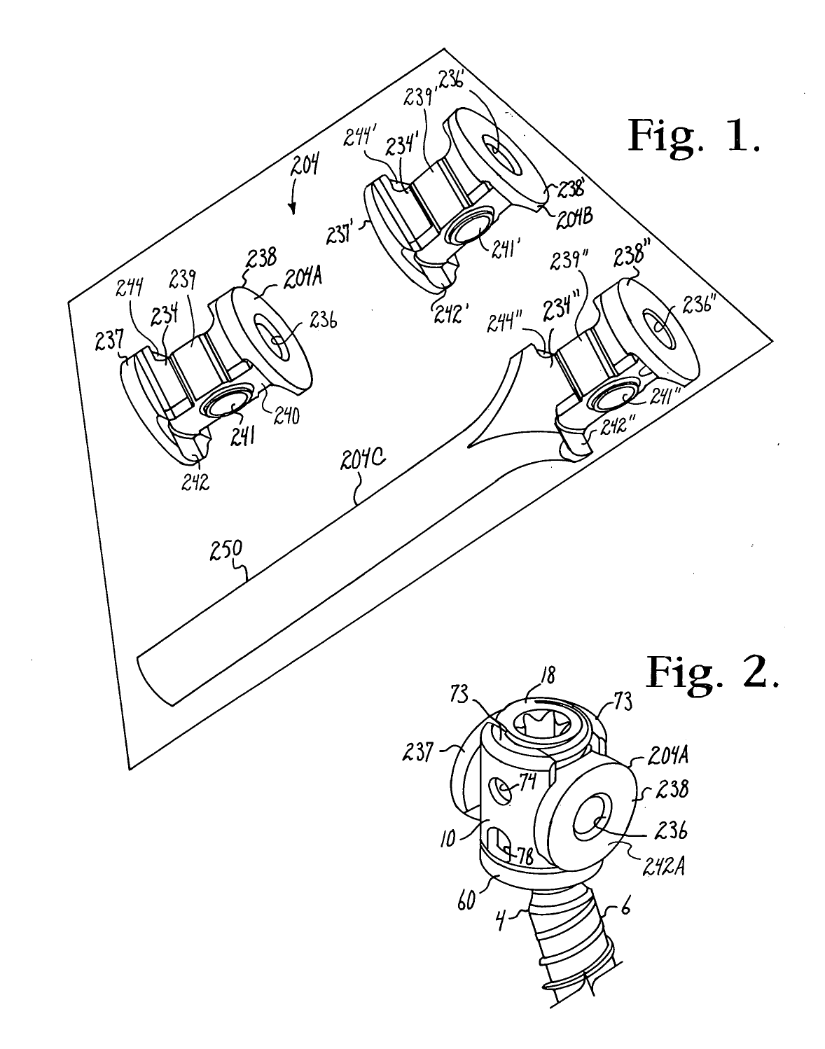

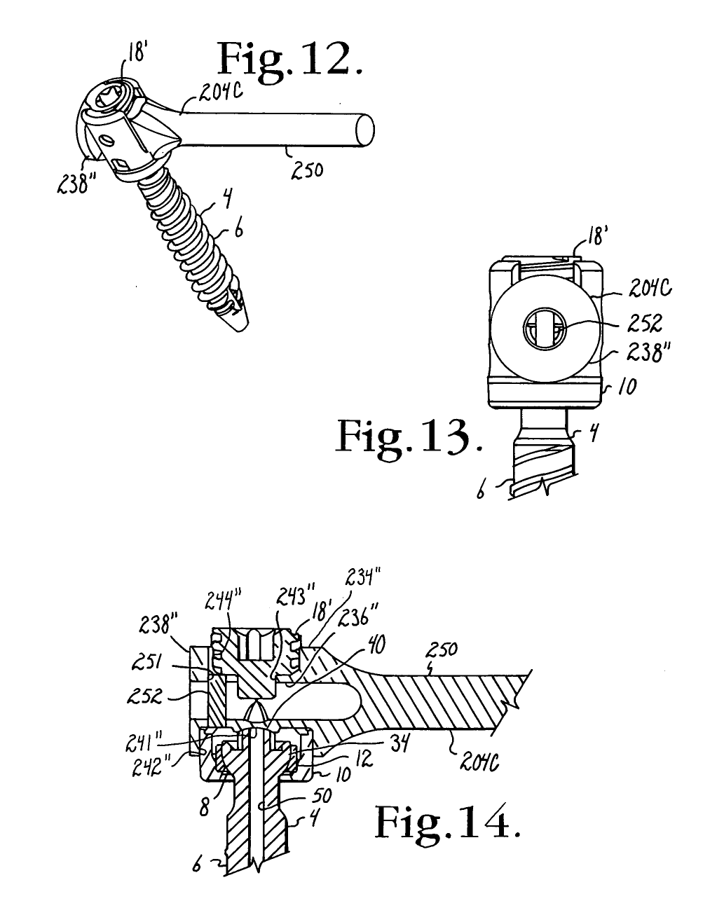

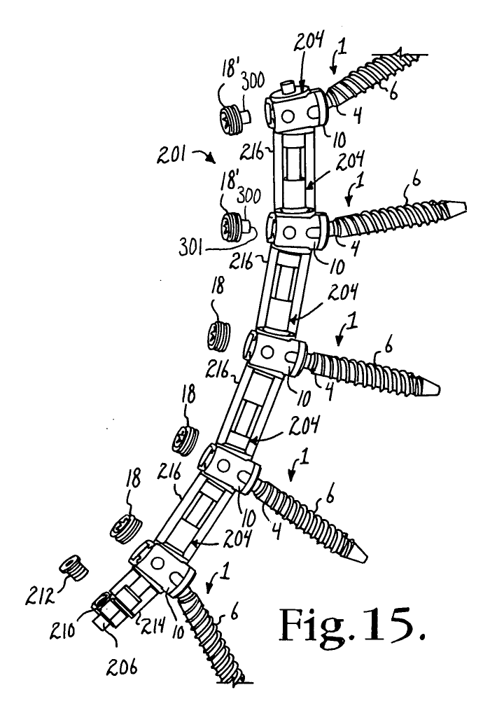

With reference to FIGS. 1-15, and particularly to FIG. 15, polyaxial bone screws, generally 1 are shown cooperating with longitudinal connecting member assemblies such as the assembly, generally 201 of the present invention, that include sleeves, generally 204 of the present invention, shown in greater detail in FIGS. 1-14. It is noted that the particular polyaxial bone screw 1 shown in FIGS. 1-15 is an example of a bone screw for use with the present invention and other types of bone screws, for example screws having top or bottom loaded shanks and/or different types of structure for capturing the shank within the receiver may be used. The bone screw 1 is described in detail in U.S. patent application Ser. No. 12/924,802 filed Oct. 5, 2010 (U.S. Patent Application Publication No. 2011/0098755 published Apr. 28, 2011, hereafter identified as the '755 publication) that is incorporated by reference herein. Briefly, with particular reference to FIG. 3, the bone screw assembly 1 includes a shank 4 having a body 6 for insertion into a vertebra, and an upper portion or head 8. The bone screw assembly further includes a receiver 10 for the pivotable housing of the shank head 8 with respect to the receiver 10, and a split ring shank retainer 12 for a pop-on or snap-on capturing of the shank head 8 within the receiver 10. The receiver is sized and shaped to mate with a closure top 18 fully described in the '755 publication that is utilized in the present invention as a cord slip or sliding closure, or with a cord fixing closure top 18′ that will be described in greater detail below. The shank head 8 has an outer partially spherical or curved surface 34 and a radiused, domed or otherwise curved top 40 located on a drive feature 41, the surface 40 being spaced from the outer spherical surface 34, the domed top 40 typically being sized and shaped for direct engagement with a rigid rod (not shown). A radius of the top 40 is typically not the same as a radius of the spherical head surface 34. In some embodiments, the top 40 and the spherical head surface 34 have the same radius. The spherical surface 34 is spaced from the shank drive feature 41. Sleeves of the present invention include a curved or radiused concave surface or surfaces shaped to advantageously frictionally mate with either the curved top surface 40 or the curved surface 34 to allow for a stable frictional engagement between the sleeve 204 (or the sleeve 1204 described below) and the shank 4 at a variety of articulations of the shank 4 with respect to the receiver 10.

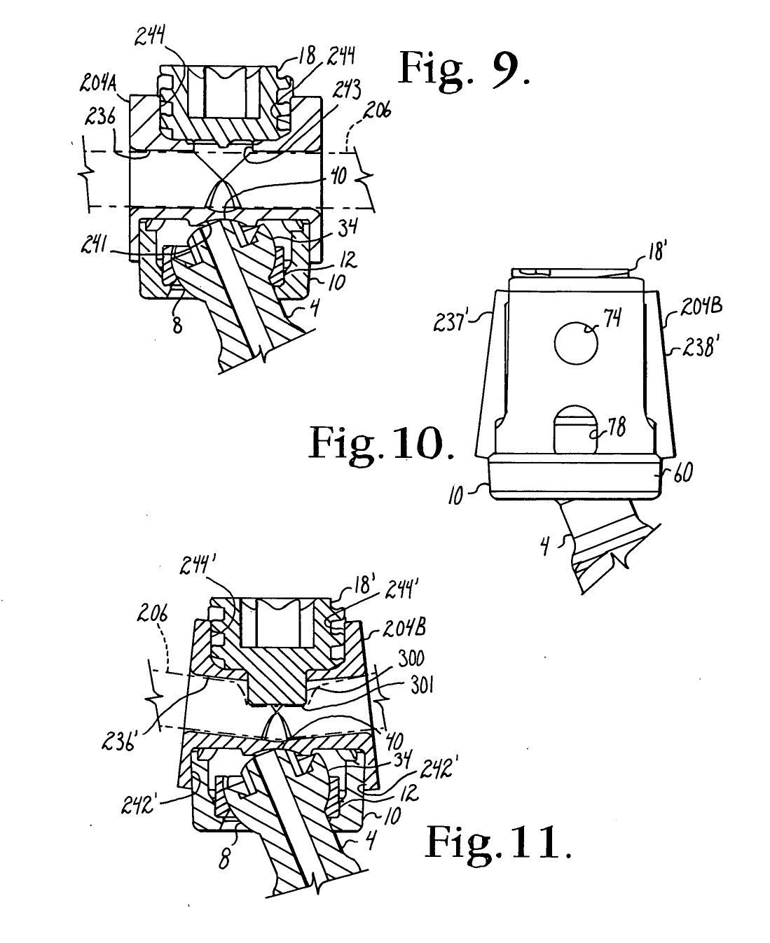

With further reference to FIG. 15, the polyaxial bone screws 1 are shown with the dynamic stabilization longitudinal connecting member assembly 201 that includes a plurality of the hard, rigid, inelastic, flanged sleeves, generally 204 through which a tensioned cord 206 ex tends. The cord 206 is shown in phantom in FIGS. 9 and 11, and see, for example, FIG. 15, that also illustrates a cooperating cord blocker or fixer 210 with a cord fixing set screw 212, an elastic end bumper 214, and elastic or inelastic spacers 216 that are each located about the cord 206 and are disposed between each pair of bone anchors 1 of the overall assembly 201. The tubular bumper 214 and tubular spacers 216 shown in FIG. 15 are transparent, allowing for viewing of the sleeves, generally 204, and the tensioned cord 206. However, it is foreseen that in other embodiments, the spacers 216 may be made of materials that may not be transparent or translucent. Cords, blockers and set screws, elastic bumpers and spacers that are the same or similar to the cord 206, blocker 210 and set screw 212, elastic bumper 214 and spacers 216 are described in detail in U.S. patent application Ser. No. 12/802,849 incorporated by reference herein (U.S. Publication No. 2010/0331887 published Dec. 30, 2010).

Also as shown in FIG. 15, two types of bone screw closures are utilized, either the slide or slipping closure top 18 described in the '755 publication or the cord gripping closure top 18′ that is described in detail in U.S. patent application Ser. No. 12/802,849 already incorporated by reference herein. The top 18′ only differs from the top 18 in that the top 18′ does not include a bottom rim or bottom point, but rather a cord penetrating extension 300 for gripping the cord 206. The extension structure 300 is illustrated has being integral with the closure, cylindrical in shape and having a planar bottom surface 301. It is noted that the extension 300 may be made of other geometric shapes and may include a more pointed surface or surfaces.

With reference to FIG. 9, the slide or slip closure top 18 engages a respective sleeve 204A but not the cord 206, allowing the cord to slip or slide within the polyaxial screw 1. With reference to FIG. 11, the gripping structure 300 of the grip closure top 18′ extends through the sleeve 204B and grips and fixes the cord 206 against a surface of the sleeve 204B and thus fixes the cord 206 in relation to the cooperating polyaxial screw 1.

With further reference to FIG. 15, tubular extensions of some of the sleeves 204 may extend into and through some or all of the spacers 216. Such spacer overlap with respect to a respective sleeve provides advantageous anti-shear support for the connecting member 201. A portion of the cord blocker 210 also extends into a bore of the bumper 214. The bumper 214 also extends about the cord 206 and is typically made from an elastomer while the outer spacers 216, although typically elastomeric, may be made from a material with a different durometer, typically (but not always) being tougher and less compressible than the material of the bumper 214. The sleeves 204 and in some embodiments the spacers 216, are typically made from a hard, non-elastic material, such as a metal or metal alloy, for example, such as cobalt chromium. Flanged portions of the sleeves 204 are located on either side of the bone screw receivers 10, the flanges abutting against the spacers 216 or the bumper 214, the flanges extending radially outwardly to an extent to fully engage ends of adjacent spacers or the bumper, resulting in a stable, secure, substantially full contact between the individual elements of the assembly 201. Furthermore, the flanges allow for assembly and dynamic setting of the connector 201 prior to implantation, if desired, with the cord 206 being placed in tension and at least the bumper 214 being placed in compression. In some embodiments of the invention, tensioning of the cord 206 and compression of the bumper 214 and optionally the spacers 216 may be performed after the assembly 201 is attached to the bone screws 1.

Sleeves 204 of the invention may be provided with or without tubular extensions, on one or both sides thereof, and with different lengths of tubular extensions, as best shown in FIG. 15. With particular reference to FIG. 1, three different types of sleeves 204, shown without tubular extensions, are illustrated. They are a parallel flanged sleeve 204A, an angled or lordotic sleeve 204B and a transition sleeve 204C that includes a rod/cord coupler.

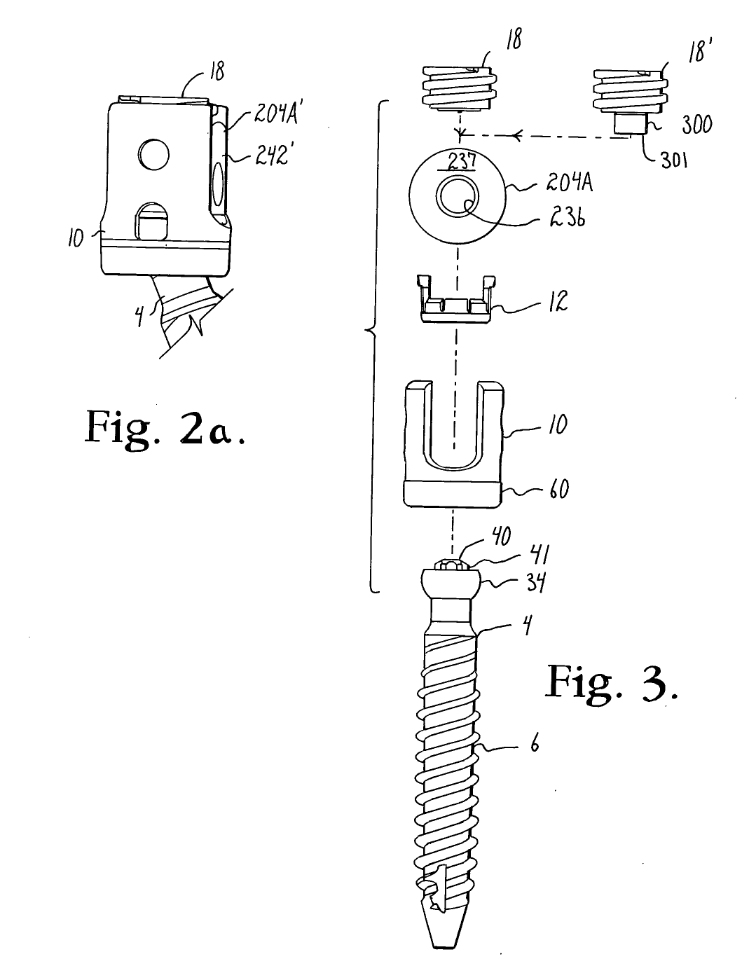

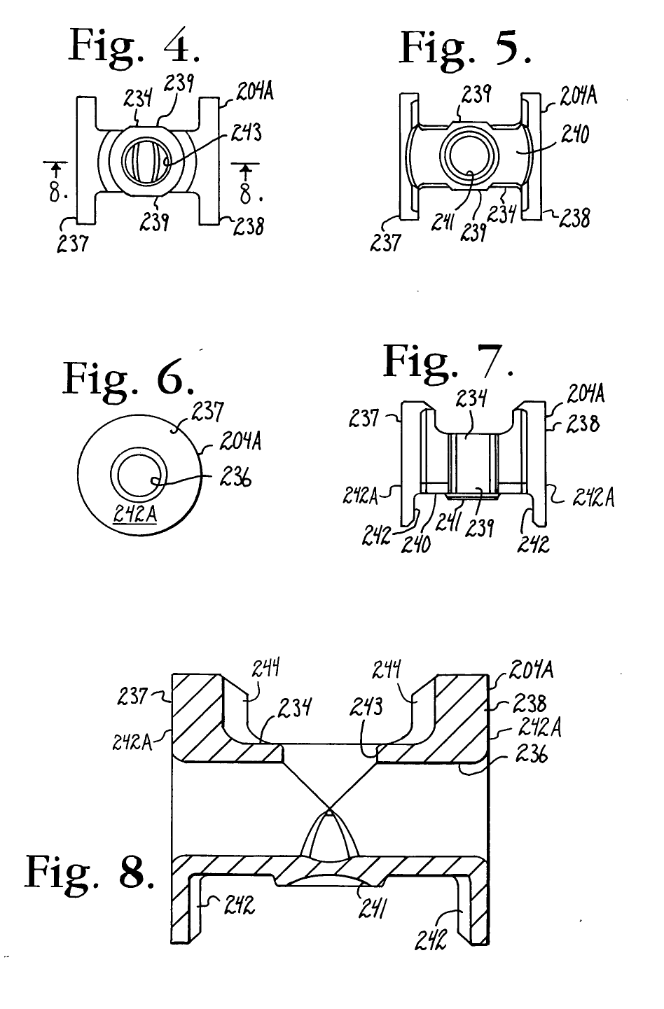

With particular reference to FIGS. 2, 3 and 9, the bone screw assembly 1 is illustrated with the sleeve 204A. With particular reference to FIGS. 4-8, the sleeve 204A further includes a body portion 234 generally sized and shaped for being received within the polyaxial bone screw 1 receiver 10 and about a cord 206. A through bore 236 extends centrally through the body portion 234, the bore 236 being sized and shaped to slidingly receive the cord 206. The body portion 234 further includes a pair of spaced radially extending flanges 237 and 238 with a partially cylindrical and partially planar body portion being located therebetween, the body portion having a slightly enlarged or protruding portion or portions illustrated as opposed faceted or partially cylindrical and partially planar extensions or body portions 239, the portions 239 sized and shaped to closely fit within inner arm surfaces of the bone screw receiver 10. The portions 239 center the sleeve within the bone screw receiver 10 and also advantageously strengthen the sleeve, resulting in better load transfer. The body 234 with centering structure 239 further includes a bottom surface 240 having a curved, concave surface, illustrated as a partially spherical surface 241 configured to closely match in size and shape and thus cooperate and frictionally engage the domed surface 40 of the shank upper portion 8 in a variety of angular configurations, as shown in FIGS. 9 and 11, for example. The illustrated surface 241 is smooth, having the same or similar radius as the domed surface 40 of the shank 4, but it is foreseen that the surface 241 (and/or the surface 40 of the shank) may be roughened or include ridges or points for penetration into the shank surface 40.

In some embodiments of sleeves of the invention, the flanges 237 and 238 may be reduced or eliminated as shown by the modified sleeve 204A′ shown in FIG. 2a having an outer planar surface 242′ being flush or nearly flush to outer surfaces of the receiver 10 that form the receiver U-shaped channel. The sleeve 204A′ is otherwise identical to the sleeve 204A shown in the other figures. The centering of the sleeve 204A′ with respect to the bone screw receiver 10 is performed by the portion or portions 239.

In the embodiment shown in the figures (other than FIG. 2a.), the flanges 237 and 238 are substantially cylindrical having opposed inner side surfaces 242 spaced and shaped for closely receiving the bone screw 1 receiver 10. Each flange also has an outer annular planar surface 242A. The illustrated flanges 237 and 238 include a lower cut-out, partially defined by the surfaces 242, allowing for a close fit between inner flange surfaces 242 and the receiver 10 at or near outer surfaces partially defining the receiver U-shaped channel and the receiver base 60. The body portion 239 as well as flange surfaces 242 may be sized and shaped to be receivable by and frictionally fixed to a variety of monoaxial or polyaxial screw heads or receivers, including the receiver 10. A bore 243 is formed in the body 234 between the flanges 237 and 238, the bore 243 being transverse to and communicating with the through bore 236. The bore 243 is sized and shaped to receive the extension structure 300 of the closure top 18′. Curved inner surfaces 244 surrounding and adjacent the bore 243 and partially defining upper portions of the flanges 237 and 238 are sized and shaped to closely receive both the closure tops 18 and 18′ as shown in FIGS. 9 and 11. The sleeve 204A is shown with the closure top 18 in FIGS. 2 and 9, and thus, as best illustrated in FIG. 9, the top 18 engages the surfaces 244 but does not extend down into the through bore 236, allowing for the cord 206 (shown in phantom) to slide freely there within. As illustrated in FIG. 11, the closure top 18′ is inserted in the sleeve 204B with the closure top body engaging surfaces 244′ as well as the extension 300 extending into and through the sleeve 204B with the surface 301 frictionally gripping a cord 206 (shown in phantom) against an internal surface defining the through bore 236′, and thus placing such cord 206 in fixed relation with the cooperating bone screw receiver 10.

The sleeves, generally 204, as well as the cord blocker 210 with set screw 212 may be made from a variety of inelastic materials, including, but not limited to metals, metal alloys, including cobalt chromium, and inelastic plastics including, but not limited to plastic polymers such as polyetheretherketone (PEEK), ultra-high-molecular weight-polyethylene (UHMWP), polyurethanes and composites, including composites containing carbon fiber and layers of different materials.

With reference to FIGS. 1, 10 and 11, the lordosing or lordotic sleeve 204B is illustrated. The sleeve 204B is identical to the sleeve 204A with the exception that flanges 237′ and 238′ are provided that slope at an angle, inwardly towards the bone screw receiver 10 as best shown in FIG. 10 and also in the assembly 201 shown in FIG. 15 that illustrates the use of a plurality of lordotic sleeves similar to the sleeve 204B, many of which further include tubular extensions on one or both ends thereof. All of the other features and structure of the sleeve 204B that are identical to the sleeve 204A are identified with the same reference numbers but with a prime symbol (“′”) located thereafter. For example, the sleeve 204B includes a body 234′, a through bore 236′, centering extension portions 239′, a bottom radiused surface 241′, lower inner flange surfaces 242′, a bore 243′ and upper inner flange surfaces 244′ that are the same or similar in form and function to the respective body 234, through bore 236, centering extension portions 239, radiused surface 241, lower inner flange surfaces 242, bore 243 and upper inner flange surfaces 244 of the sleeve 204A previously described herein.

With reference to FIG. 1 and FIGS. 12-14, the sleeve and rod/cord coupler 204C includes sleeve body 234″, a partial through bore 236″, a single flange 238″, centering extension portions 239″, a bottom radiused surface 241″, lower inner flange surfaces 242″, a bore 243″ and upper inner flange surfaces 244″ that are substantially similar in form and function to the respective sleeve body 234, bore 236, flange 238, centering portions 239, radiused surface 241, lower inner flange surfaces 242, bore 243 and upper inner flange surfaces 244 of the sleeve 204A previously described herein. At an end opposed to the flange 238″, the body portion 234″ is integral with an elongate solid rod portion 250. Also, formed in the body portion 234″ is an aperture or through bore 251 transverse to and communicating with the bore 236″, the through bore 251 being sized and shaped to closely receive a cord holding pin 252. The pin 252, if used, extends completely through the cord 206, independently fixing the cord 206 to the sleeve 204C. Alternatively, in some embodiments of the invention, the pin 252 is not used and the closure top 18′ is inserted within a bore 243″ of the sleeve/coupler 204C to fix the cord 206 to the sleeve 204C. Although FIG. 14 illustrates using both the closure top 18′ and the pin 252, this is for the purpose of illustration and is not necessary for the practice of the invention. In the illustrated embodiment, the bores 243″ and 251 are substantially parallel to one another. The rod portion 250 may be provided in a variety of lengths (or cut to length) to cooperate with one or more bone screws to provide a rigid support end to a dynamic assembly, such as the assembly 201 shown in FIG. 15.

The assembly 201 may be assembled as follows: First, after the bone screws 1 are implanted, the distance between the screws is measured. Thereafter, the spacers 216 are cut to a desired length based upon the measurement made between the bone screws. Because the sleeves 204 are made from a hard material, typically a metal or metal alloy, if it is desired to use sleeves with tubular extensions as shown in FIG. 15, it is not practical to cut the tubular portions to a desired length during the surgical procedure. Therefore, a variety of sleeves 204 are typically provided to end users having at least three different tube portion lengths.

With particular reference to FIG. 15, sleeves 204, spacers 216, cord blockers 210 and, if desired, a rod/sleeve coupler 204C are fed onto a cord 206 in a desired order to result in the assembly of FIG. 15 and in a manner as described in greater detail in the patent application Ser. No. 12/802,849 incorporated by reference herein. It is noted that the cord 206 is typically much longer than shown in the drawing figures and then cut to length near an end thereof after being fully assembled with the remaining elements of the assembly 201, tensioned and fixed to the blocker 210. In some embodiments of the invention, single blockers, bumper/blocker combinations or rod/cord couplers (or various different combinations thereof) may be placed on either end of the assembly and the cord pre-tensioned before the assembly is implanted in and between the already implanted bone screws 1. In other embodiments, such as the assembly shown in FIG. 15, a loosely assembled connector may be placed in contact with and between the implanted bone screws 25, with the set screw 212 engaged with the cord 206 enough to prevent the elements from slipping off one end of the cord 206. But, unlike the illustration of FIG. 15, the cord 206 would not yet be tensioned and thus the individual elements would most likely be more spread apart along the cord more than is illustrated in the drawing figure. Also, the cord 206 would be much longer at this time so that the cord may be grasped and tensioned after the assembly is fixed to the bone screws 1.

The assembly 201 is implanted by inserting each sleeves 204 into to one of the bone screws 1. Closure tops 18 or 18′ are then inserted into and advanced between the arms of the bone screw receiver 10 so as to bias or push against the respective sleeves 204. A driving tool (not shown) is inserted into each closure drive to rotate and drive the respective closure top 18 or 18′ into the respective receiver 10. Each shank dome surface 40 is engaged by the cooperating sleeve surface 241, 241′ or 241″ and pushed downwardly when the closure top 18 or 18′ pushes downwardly on the sleeve 204A, B or C. The downward pressure on the shank 4 in turn urges the shank spherical surface 34 into locking engagement with the retainer 12 that in turn expands into locking engagement with the receiver 10. A tensioning tool (not shown) known in the art may then be used to pull upon and put tension on the cord 206. It is noted that in the particular embodiment shown in FIG. 15, two gripping closures 18′ are shown. Thus, it may be desirable to lock only one of the closure 18′ initially and thereafter lock the other after tensioning, or alternatively perform more than one tensioning step. The cord 206 is preferably tensioned until the bumper compresses and then the set screw 212 is rotated and driven into the blocker 210 and up against the cord 206 using a driving tool (not shown) engaged with an inner drive of the screw 212. The blocker 210 advantageously includes opposed planar sides allowing for the placement of a counter-torque tool for holding the blocker during tensioning and fixing of the cord 206 within the blocker. As explained in U.S. patent application Ser. No. 12/802,849, the set screw 212 and blocker 210 combination includes a limited travel feature such that the set screw is locked into place at a location that firmly holds but does not damage the cord 206. The cord 206 is ultimately trimmed to a desired length close to each end of the connector 201.

The assembly 201 is thus substantially dynamically loaded and oriented relative to the cooperating vertebra, providing relief (e.g., shock absorption) and protected movement with respect to flexion, extension, distraction and compressive forces placed on the assembly 201 and the connected bone screws 1. In some embodiments of a connecting member according to the invention, the sleeve and rod combination 204C may be used at one end (or both ends) of the assembly to provide a hard, non-elastic elongate portion for attachment to an additional bone screw or screws, if needed, to provide a connecting member with both dynamic, elastic segments as well as a longer rigid inelastic segment.

If removal of the assembly 201 from any of the bone screw assemblies 1 is necessary, or if it is desired to release the assembly at a particular location, disassembly is accomplished by using the driving tool (not shown) with a driving formation cooperating with the closure structure 18 or 18′ internal drive to rotate and remove the closure structure from the respective receiver 10. Disassembly is then accomplished in reverse order to the procedure described previously herein for assembly.

Eventually, if the spine requires more rigid support, the connecting member assembly 201 according to the invention may be removed and replaced with another longitudinal connecting member, such as a solid rod or bar, having the same width or diameter as body portions of the sleeves 204, utilizing the same receivers 10 and the same or similar closure structures 18. Alternatively, if less support is eventually required, a less rigid, more flexible assembly, for example, an assembly having spacers 216 and a bumper or bumpers 214 made of a softer more compressible material than the spacer and bumper being replaced thereby, also utilizing the same bone screws 1 and the closures 18′ as well as the closure 18.

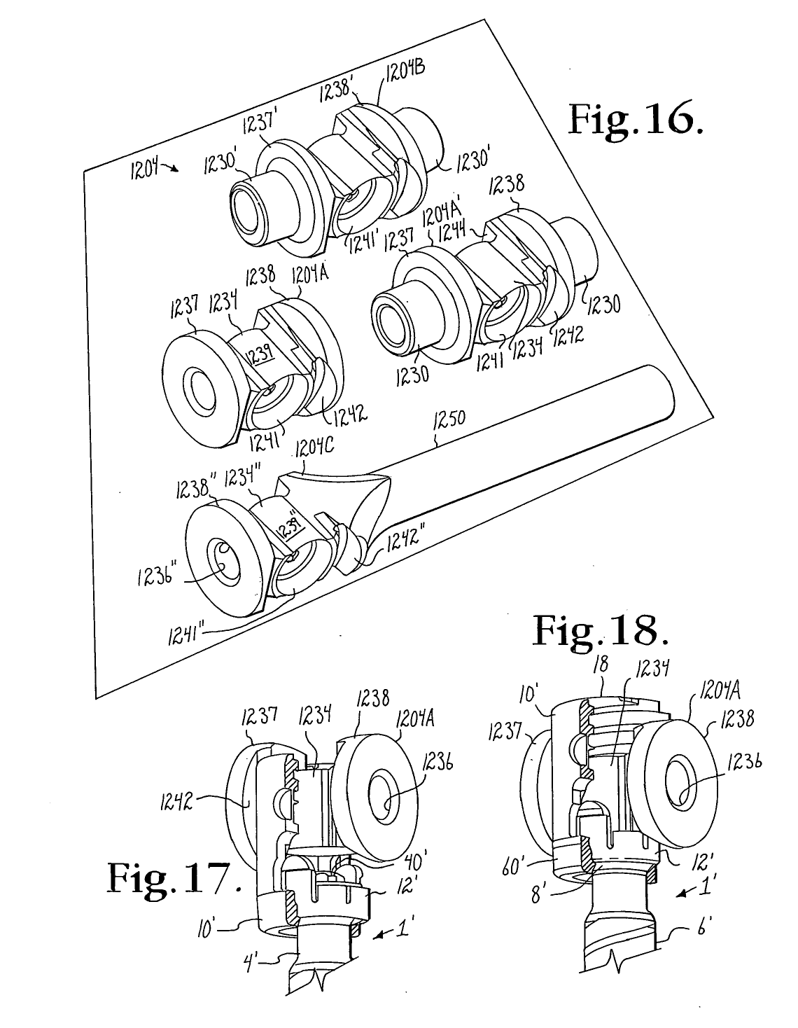

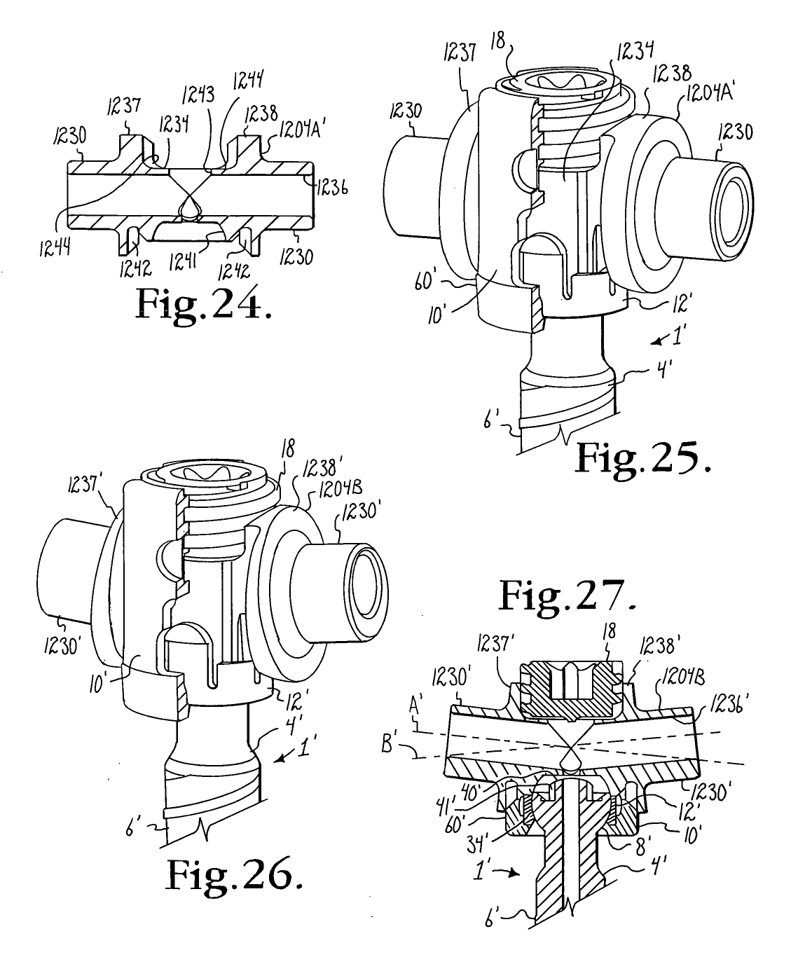

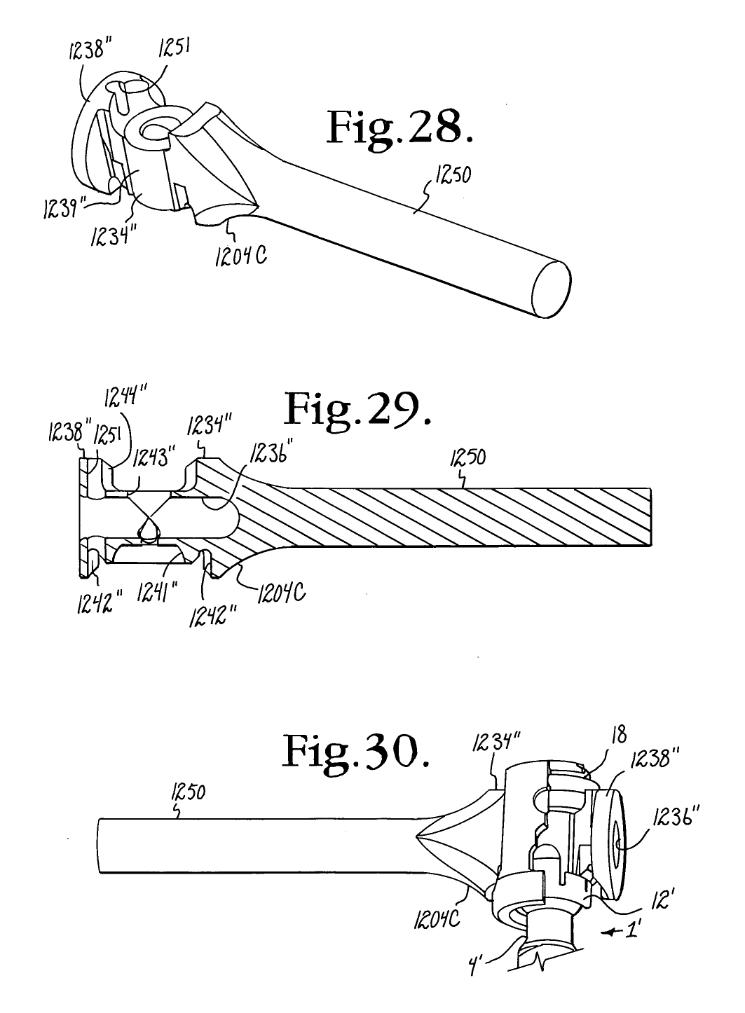

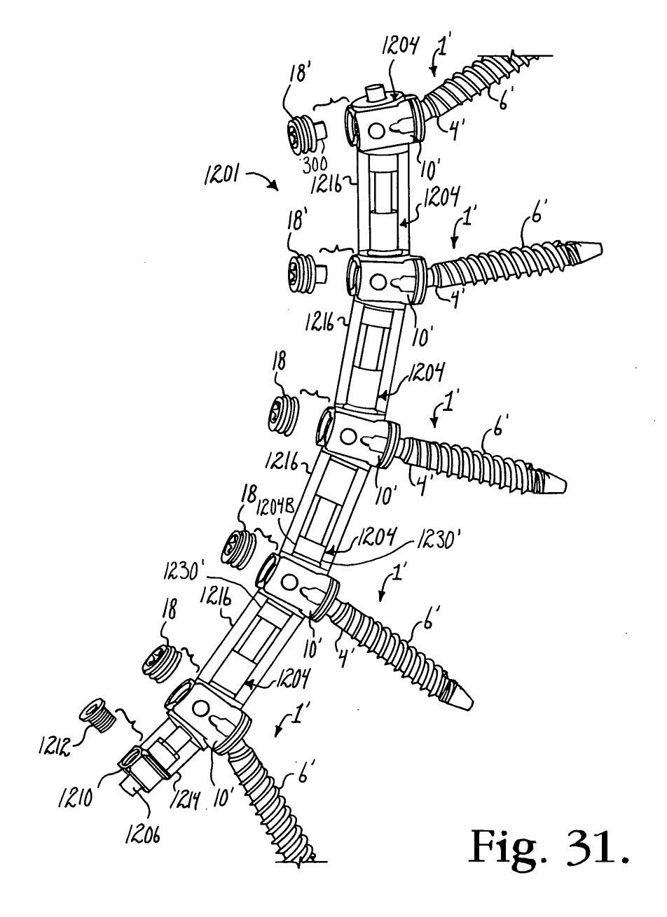

With reference to FIGS. 16-34, another embodiment of connecting member sleeves according to the invention, generally 1204, is shown. The sleeves 1204 are substantially similar to the sleeves 204 previously described herein and with the exception of the size and placement of a radiused lower surface that engages a larger spherical surface of a cooperating bone anchor shank (e.g., the spherical surface 34 of the shank 4 or surface 34′ of a shank 1′) than the domed surface 40 previously described herein with respect to the bone screw assembly 1. With particular reference to FIG. 31, polyaxial bone screws, generally 1′ are shown cooperating with a longitudinal connecting member assembly of the invention, generally 1201, that includes the sleeves, generally 1204 of the present invention that are shown in greater detail in FIGS. 16-30 and 34. The polyaxial bone screw 1′ shown in FIGS. 16-31 is similar, but not identical to the bone screw 1 previously described herein. The screw 1′ is also described in detail in U.S. patent application Ser. No. 12/924,802, already incorporated by reference herein. Briefly, with particular reference to FIGS. 17, 18 and 19, the bone screw assembly 1′ includes a shank 4′ having a body 6′ for insertion into a vertebra, and an upper portion or head 8′. The bone screw assembly further includes a receiver 10′ for pivotable housing the shank head 8′ with respect to the receiver 10′ and a split ring shank retainer 12′ for capturing the shank head 8′ within the receiver 10′. The receiver is sized and shaped to mate with the slip closure top 18 or the grip closure top 18′ fully described in the '755 publication and previously described hereon with respect to the bone screw 1. The shank head 8′ has an outer partially radiused or spherical surface 34′ and a radiused, domed or otherwise curved top 40′ located on a drive feature 41′, the same or substantially similar to the respective head outer surface 34, domed top 40 and drive feature 41 previously described herein with respect to the bone screw 1.

With further reference to FIG. 31, the polyaxial bone screws 1′ are shown with the dynamic stabilization longitudinal connecting member assembly 1201 that includes a plurality of the hard, inelastic, flanged sleeves, generally 1204 through which a tensioned cord 1206 extends. The cord 1206 is substantially the same or similar to the cord 206 previously described herein. The assembly further includes a cord blocker 1210 with set screw 1212, a bumper 1214 and spacers 1216 substantially the same or similar in form and function to the respective blocker 210, set screw 212, bumper 214 and spacers 216 previously described herein with respect to the assembly 201. Tubular extensions of some of the sleeves 1204 are shown extending into and through some of the spacers 1216. Flanged portions of the sleeves 1204 are located on either side of the bone screw receivers 10′, the flanges abutting against the spacers 1216 or the bumper 1214, the flanges extending radially outwardly to an extent to fully engage ends of adjacent spacers or the bumper, resulting in a stable, secure, substantially full contact between the individual elements of the assembly 1201. Furthermore, the flanges allow for assembly and dynamic setting of the connector 1201 prior to implantation, if desired, with the cord 1206 being placed in tension and at least the bumper 1214 being placed in compression, such arrangements having blockers, rod/cord couplers or other fixing structure attached to the cord at either end of the assembly. In some embodiments of the invention, tensioning of the cord 1216 and compression of the bumper 1214, if used and optionally the spacers 1216 may be performed after the assembly 1201 is attached to the bone screws 1′.

Sleeves 1204 of the invention may be provided with or without tubular extensions, on one or both sides thereof, and with different lengths of tubular extensions, as best shown in FIG. 31. With particular reference to FIG. 16, three different types of sleeves 1204 are shown. They are a parallel flanged sleeve 1204A, as well as a parallel flanged sleeve 1204A′ having tubular extensions 1230, an angled or lordotic sleeve 1204B, shown with tubular extensions 1230 and a transition sleeve 1204C that includes a rod/cord coupler.

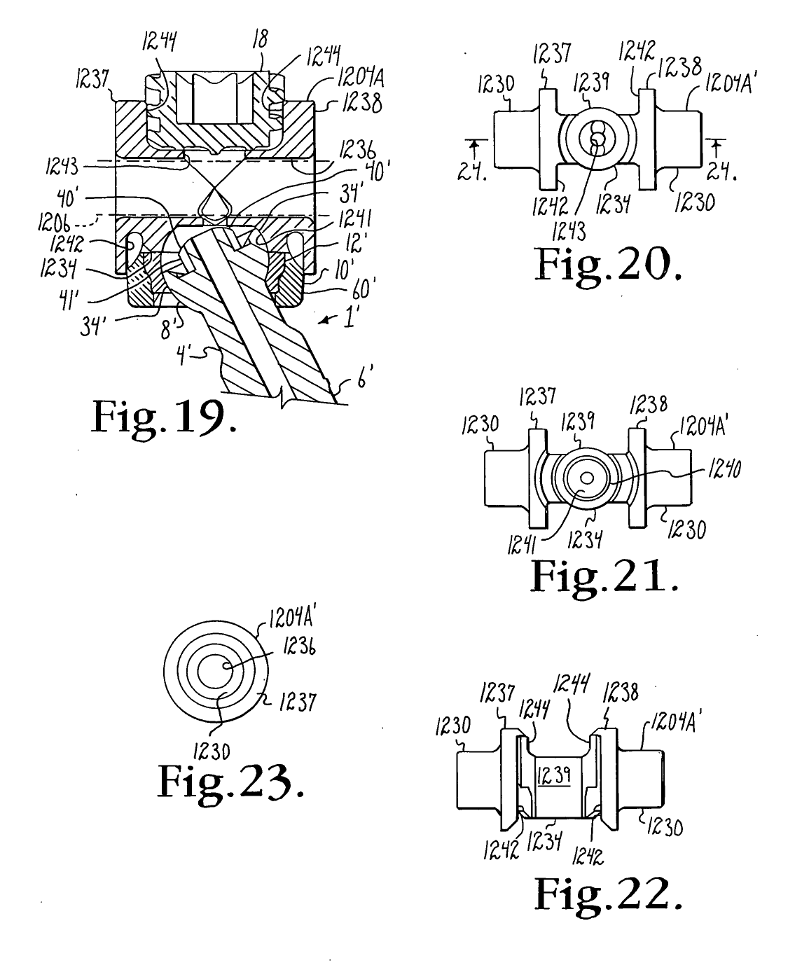

With particular reference to FIGS. 17-19, the bone screw assembly 1′ is illustrated with the sleeve 1204A. Because the sleeves 1204A and 1204A′ are identical with the exception that the sleeve 1204A′ further includes the cylindrical tubes 1230 on either side thereof, the same reference numerals are otherwise used for the sleeves 1204A and 1204A′. With further reference to FIGS. 19-25, the sleeves 1204A and 1204A′ further each include a body portion 1234 generally sized and shaped for being received within the polyaxial bone screw receiver 10′ and about a cord 1206. A through bore 1236 extends centrally through the body portion 1234, the bore 1236 being sized and shaped to slidingly receive the cord 1206. The body portion 1234 further includes a pair of spaced radially extending flanges 1237 and 1238 with a partially cylindrical and partially planar body portion being located therebetween, the body portion having a slightly enlarged or protruding portion or portions illustrated as partially cylindrical surface portions 1239 sized and shaped to closely fit and thus center the sleeve at a desired location within the inner arm surfaces of the bone screw receiver 10′. In addition to generally centering the sleeve within a bone screw receiver or other bone anchor, the portions 1239 advantageously strengthen the sleeve, resulting in better load transfer. The body 1234 with centering structure 1239 further includes a bottom surface 1240 having a curved, concave surface, illustrated as a spherical surface 1241 configured to closely cooperate and frictionally engage the spherical surface 34′ of the shank upper portion 8 at a variety of angles or articulations of the shank with respect to the sleeve, one of which is shown, for example, in FIG. 19. The illustrated surface 1241 has the same or substantially similar radius as the shank surface 34′ and is smooth. It is foreseen that the surface 1241 may be roughened or include ridges or points for penetration into the shank surface 34′.

Similar to what is shown in FIG. 2a with respect to the sleeve 204A′, in some embodiments, the flanges 1237 and 1238 may be reduced or eliminated as the centering of the sleeve with respect to the bone screw receiver 10′ may be performed solely by the portion or portions 1239. In the illustrated embodiment, the flanges 1237 and 1238 are substantially cylindrical having opposed lower curved inner side surfaces 1242 spaced for closely receiving outer surfaces of the bone screw receiver 10′ near the base 60′ thereof. The illustrated flanges 1237 and 1238 each include a lower cut-out, partially formed by the inner side surfaces 1242 that allows for a close fit between the inner flange surfaces 1242 and the receiver 10′ surfaces. The body portion 1239 as well as flange surfaces 1242 may be sized and shaped to be receivable by and frictionally mated to a variety of monoaxial or polyaxial screw heads or receivers, including, but not limited to the receiver 10′ as well as the receiver 10 previously discussed herein. A bore 1243 is formed in the body 1234 between the flanges 1237 and 1238, the bore 1243 being transverse to and communicating with the through bore 1236. Upper curved surfaces 1244 surrounding and adjacent the bore 1243 and partially defining inner upper portions of the flanges 1237 and 1238 are sized and shaped to closely receive both the closure tops 18 and 18′ as shown, for example, in FIG. 19. With specific reference to FIG. 19, the non-gripping closure top 18 engages the surfaces 1244 but does not extend through the bore 1243 and thus does not extend down into the through bore 1236, allowing for the cord 1206 (shown in phantom) to slide freely within the bore 1236. Similar to what is shown with respect to the sleeves 204, as illustrated in FIG. 11, when the closure top 18′ is inserted into any of the sleeves 1204 with the extension 300 extending into and through the sleeve, the surface 301 frictionally grips the cord 1206 against an internal surface defining the through bore 1236, and thus places such cord 1206 in fixed relation with the cooperating bone screw receiver 10′.

The sleeves, generally 1204, as well as the cord blocker 1210 with set screw 1212 may be made from a variety of inelastic materials, including, but not limited to metals, metal alloys, including cobalt chromium, and inelastic plastics including, but not limited to plastic polymers such as polyetheretherketone (PEEK), ultra-high-molecular weight-polyethylene (UHMWP), polyurethanes and composites, including composites containing carbon fiber and layers of different materials.

With reference to FIGS. 26 and 27, the lordosing or lordotic sleeve 1204B is illustrated. The sleeve 1204B is identical to the sleeve 1204A′ with the exception that flanges 1237′ and 1238′ are provided that slope at an angle, inwardly towards the bone screw receiver 10′ as best shown in FIG. 27 and also in the assembly 1201 shown in FIG. 31 that illustrates the use of a plurality of lordotic sleeves similar to the sleeve 1204B, some of which further include tubular extensions 1230, 1230′, no extensions, and/or longer sleeve extensions or combinations of short, long and/or no extensions on the ends thereof. Unlike the sleeves 1204A and 1204A′ that have the inner bore 1236 extending along a single central longitudinal axis, as best shown in FIG. 27, the sleeves 1204B form an inner bore 1236′ that is defined by an intersection of two axes A′ and B′, the intersection located centrally within the sleeve.

With reference to FIG. 16 and FIGS. 28-30, the sleeve and rod/cord coupler 1204C includes a sleeve body 1234″, a partial through bore 1236″, a single flange 1238″, centering extension portions 1239″, a bottom radiused surface 1241″, lower inner flange surfaces 1242″, a bore 1243″ and upper inner flange surfaces 1244″ that are substantially similar in form and function to the respective sleeve body 1234, bore 1236, flange 1238, centering portions 1239, radiused surface 1241, lower inner flange surfaces 1242, bore 1243 and upper inner flange surfaces 1244 of the sleeves 1204A and 1204A′ previously described herein. At an end opposed to the flange 1238″, the body portion 1234″ is integral with an elongate solid rod portion 1250. Also, formed in the body portion 1234″ is an aperture or through bore 1251 disposed transverse to and communicating with the bore 1236″, the through bore 1251 sized and shaped to closely receive a cord holding pin (not shown) the same or similar to the pin 252 illustrated in FIGS. 13 and 14. The pin, if used, extends completely through the cord 1206, independently fixing the cord 1206 to the sleeve 1204C. Alternatively, in some embodiments of the invention, the pin 1252 is not used and a closure top 18′ may be inserted within the bore 1243″ of the sleeve/coupler 1204C to fix the cord 1206 to the sleeve 1204C. In the illustrated embodiment, the bores 1243″ and 1251 are substantially parallel to one another. The rod portion 1250 may be provided in a variety of lengths (or cut to length) to cooperate with one or more bone screws to provide a rigid support end to a dynamic assembly, such as the assembly 1201 shown in FIG. 31.

In use, sleeves generally 1204, of the invention are assembled into a longitudinal connecting member, such as the member 1201 illustrated in FIG. 31 in a manner identical or substantially similar to what has been previously described herein with respect to the assembly 201 shown in FIG. 15. Disassembly and replacement with a harder, stiffer connector or a softer connector is also accomplished in a manner the same or similar to what has already been described herein with respect to the assembly 201. Briefly, the assembly 1201 is implanted by inserting each of the sleeves 1204 into to one of the bone screws 1′ (or the bone screws 1 or other cooperating bone anchors). Closure tops 18 or 18′ are then inserted into and advanced between the arms of the bone screw receiver 10′ so as to bias or push against the respective sleeves 1204. A driving tool (not shown) is inserted into each bone screw closure drive to rotate and drive the respective closure top 18 or 18′ into the respective receiver 10′. Each shank head partially spherical surface 34′ is engaged by the cooperating sleeve surface 241, 241′ or 241″ and pushed downwardly when the closure top 18 or 18′ pushes downwardly on the sleeve 1204A, 1204A′, 1204B or 1204C. The downward pressure on the shank 4′ in turn urges the shank spherical surface 34′ into locking engagement with the retainer 12′ that in turn expands into locking engagement with the receiver 10′. A tensioning tool (not shown) known in the art may then be used to pull upon and put tension on the cord 1206 for the first time, or may be used to place additional tension on an already pre-tensioned connecting member assembly that includes a blocker 1210 or blocker/bumper 1214 combination at either end thereof, or a blocker or blocker/bumper at one end thereof and a rod/cord coupler 1204C at an opposite end thereof.

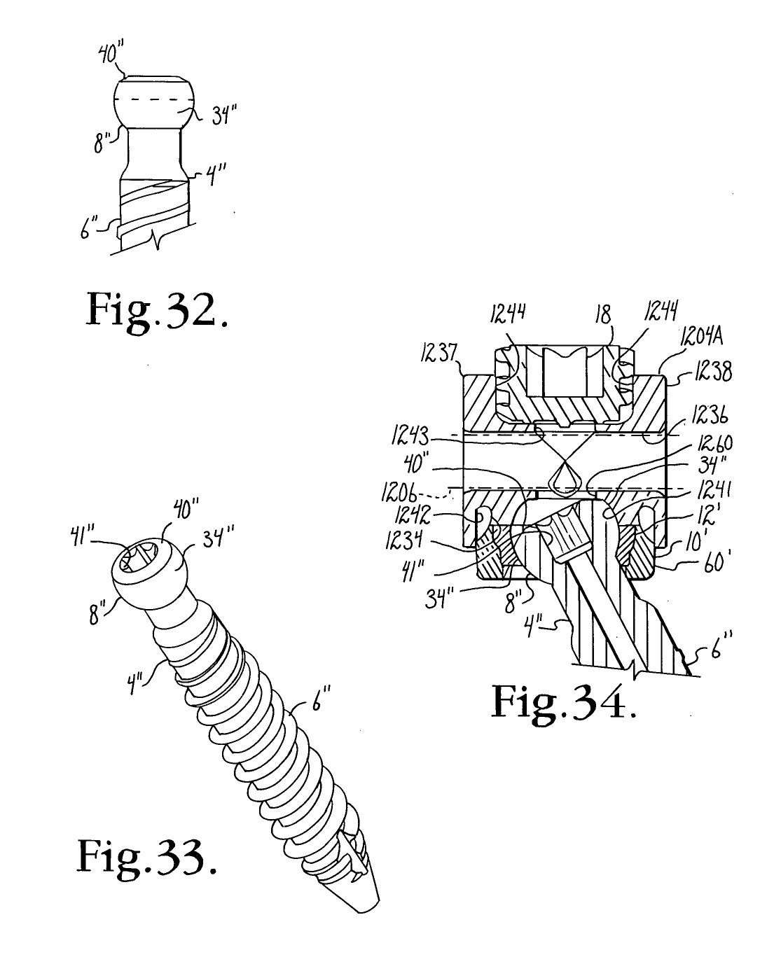

With reference to FIGS. 32-34, an alternative bone screw shank 4″ is illustrated that includes an internal drive feature 41″ in lieu of the domed drive 41′ shown in FIGS. 19 and 27. The shank 4″ otherwise includes a body 6″ and an upper portion or head 8″ with an outer spherical surface 34″ that is the same or substantially similar in form and function to the respective body 6′, head 8′ and spherical surface 34′ previously described herein with respect to shank 4′ of the bone screw assembly 1′. The shank 4″ further includes a rim or frusto-conical surface 40″ sized and shaped to allow for adequate clearance between the shank head 8″ and the sleeve, generally 1204 during pivoting of the shank 4″ with respect to the receiver 10.

In other embodiments, the surface 34″ may extend to the drive feature 41″ and the concave spherical lower surface 1241 of the sleeve may be extended in a direction toward the bore 1236 to provide greater clearance for the spherical bone shank head during pivoting thereof. In FIG. 34, the shank 4″ is shown attached to and cooperating with the receiver 10′ and the retainer 12′ of the bone screw 1′ and also with a slightly modified version of the sleeve 1204A, as well as the closure top 18 in a manner the same or substantially similar to what is shown in FIG. 19 and described previously herein with respect to the sleeve 1204A and the bone screw assembly 1′. Because the sleeve 1204A includes the radiused surface 1241 that engages the head spherical surface 34″ (or the surface 34′ of the bone screw assembly 1′ or the surface 34 of the bone screw assembly 1), a variety of bone screws having shanks with different driving and bone screw shank capturing and securing features may be used with sleeves of the invention. Thus the bone screw assembly shown in FIG. 34 that has the shank 4″ that includes the internal drive feature 41″ should not be considered limiting, but rather as an additional embodiment of a cooperating bone screw of which there is a wide variety.

The illustrated drive feature or imprint 41″ is counter sunk and includes a stepped or graduated annular seating surface or base. The base surface could be substantially planar. The illustrated internal drive feature 41″ is a star shaped or multi-lobular aperture designed to receive a tool (not shown) of an Allen wrench type, into the aperture 41″ for rotating and driving the bone screw shank 4″ into the vertebra. It is foreseen that such an internal tool engagement structure may take a variety of tool-engaging forms and may include one or more apertures of various shapes, such as a pair of spaced apart apertures or a hex shaped aperture.

The sleeve 1204A illustrated in FIG. 34 is modified slightly to provide a lower aperture 1260 being sized and shaped to be large enough to receive the driving tool for the shank drive feature 41″. The upper aperture 1243 is also large enough to receive the shank driving tool. In operation, the driving tool (not shown) may therefore be received through both apertures 1243 and 1260 of the sleeve 1204A, followed by being received in the internal drive feature 41″ and being seated at the base thereof, engaging the faces of the drive feature 41″ for both driving and rotating the shank body 6″ into a vertebra. The receiver 10′ already equipped with the retainer and the sleeve 1204A, or for example, the sleeve 294A′ shown in FIG. 2a having the surfaces 242′ that are flush with surfaces of the receiver 10, may be advantageously provided to a user as a pre-assembled unit. The receiver with sleeve may therefore be mounted on the shank head 8″ before or after the shank 4″ is driven into the vertebra. In the illustrated embodiment, the driving tool can readily extend through the sleeve at the apertures 1243 and 1260, for example and then into the drive feature 41″ axially aligned with the receiver axis, when the shank 4″, retainer 12′, sleeve (204A′ or 1204A, modified with a wide enough aperture 1260 shown in FIG. 34) and receiver 10′ combination is driven into the vertebra. The cord 1206 may thereafter be threaded through the aperture 1236 and either a sliding closure 18 or a gripping closure 18′ may be used. If a gripping closure 18′ is used, some of the cord may be pressed into the aperture 1260 which is not problematic as the cord is adequately gripped and held at edges defining the aperture 1260 as well as by other surfaces defining the closure 18′ and the bore 1236.

It is to be understood that while certain forms of the present invention have been illustrated and described herein, it is not to be limited to the specific forms or arrangement of parts described and shown.

What is claimed and desired to be secured by Letters Patent is as follows:

1. In a medical implant assembly having at least a pair of bone anchors cooperating with a longitudinal connecting member having a tensioned cord and a spacer located between the first and second bone anchors, the cord extending through the spacer, the bone anchors having opposed upstanding arms,
the improvement wherein at least one of the bone anchors is a polyaxial bone anchor having a shank pivotable with respect to a receiver, the shank having a first radiused surface, and further comprising:
at least one inelastic sleeve for attachment to the polyaxial bone anchor, the sleeve having a first through bore sized and shaped for slidably receiving the tensioned cord, a first aperture formed in the sleeve substantially transverse to the first through bore, the first aperture sized and shaped for receiving a portion of an optional cord gripping closure top and a first body portion sized and shaped for being closely received between the bone anchor arms, the sleeve having a second surface sized and shaped for directly engaging the shank first radiused surface at a variety of angular orientations of the shank with respect to the sleeve.

2. The improvement of claim 1 wherein the sleeve is a first sleeve and the polyaxial bone anchor is a first polyaxial bone anchor and further comprising:
a) a second polyaxial bone anchor having a third radiused surface; and
b) a second inelastic sleeve for attachment to the second bone anchor, the second sleeve having a second through bore sized and shaped for slidably receiving the tensioned cord, a second aperture formed in the sleeve substantially transverse to the second through bore, the second aperture sized and shaped for receiving a portion of a second optional cord gripping closure top and a second body portion sized and shaped for being closely received between the second bone anchor arms, the second sleeve having a fourth surface sized and shaped for directly engaging the third radiused surface at a variety of angular orientations of the second shank with respect to the second sleeve.

3. The improvement of claim 2 comprising at least a first closure top having an extended portion in gripping contact with the cord and fixed to one of the first and second sleeves.

4. The improvement of claim 2 comprising at least a second closure top having a surface in gripping contact with one of the first and second sleeves, the closure top being spaced from the cord.

5. The improvement of claim 1 wherein the sleeve includes an extension structure slidingly received over the cord and within the spacer.

6. The improvement of claim 1 wherein the shank first surface is a convex top surface of the shank and the sleeve second surface is concave, the first and second surfaces having substantially the same radius.

7. The improvement of claim 6 wherein the top surface is located on a drive feature of the shank.

8. The improvement of claim 1 wherein the shank has an upper portion with a partially spherical surface, the spherical surface is the first radiused surface and the sleeve second surface is concave, the first and second surfaces having substantially the same radius.

9. The improvement of claim 8 wherein the partially spherical surface is spaced from a drive feature of the shank.

10. The improvement of claim 2 wherein at least one of the first and second sleeves includes an outer flange abutting against an outer surface of the spacer.

11. The improvement of claim 10 wherein the outer flange is a first outer flange and further comprising a second outer flange substantially parallel to the first outer flange, each flange being disposed outside of the cooperating bone anchor.

12. The improvement of claim 10 further comprising at least one tubular structure extending outwardly from the flange.

13. The improvement of claim 10 wherein the outer flange is a first outer flange and further comprising a second outer flange disposed at an obtuse angle with respect to the first outer flange, each flange being disposed outside of the cooperating bone anchor.

14. The improvement of claim 13 further comprising a first tubular structure having a first central axis extending outwardly from the first flange and a second tubular structure having a second central axis extending from the second flange, the first and second axes forming an obtuse angle.

15. The improvement of claim 1 wherein the sleeve has an outer surface substantially flush with an outer surface of the bone anchor.

16. In a medical implant assembly having at least first and second polyaxial bone anchors cooperating with a longitudinal connecting member, the improvement wherein the connecting member comprises:
a) a first hard, non-elastic sleeve for attachment to the first bone anchor, the first sleeve having a first radiused surface sized and shaped for frictional mating with the first polyaxial bone anchor shank, the shank having a mating surface with a second radius substantially similar to the first radiused surface;
b) a second non-elastic sleeve for attachment to the second bone anchor;
c) a spacer located between the first and second bone anchors, the spacer engaging the first and second sleeves; and
d) a tensioned cord slidingly received within the first and second sleeves and the spacer, the cord being in one of a fixed and slidable relation with respect to the first and second sleeves.

17. The improvement of claim 16 wherein at least one of the first and second sleeves includes an extension structure slidingly received over the cord and slidingly received within the spacer.

18. The improvement of claim 16 wherein the shank mating surface is at least one of a top surface of the shank located on a drive feature and a partially spherical surface spaced from the drive feature.

19. The improvement of claim 16 wherein at least one of the sleeves has an aperture formed therein sized and shaped to receive a portion of a closure top, the closure top portion being fixed against the cord.

20. In a medical implant assembly having at least first and second bone anchors cooperating with a longitudinal connecting member having a tensioned cord, the improvement comprising:
a) at least one non-elastic sleeve for attachment to the first bone anchor, the sleeve having a body portion for slidingly receiving the cord therethrough, the body portion sized and shaped for being closely received between opposed arms of the first bone anchor, the body portion having a concave surface sized and shaped for direct contact and frictional mating with a convex surface of a shank of at least one of the bone anchors;
b) a spacer located between the first and second bone anchors, the cord extending through the spacer and held in tension at least first and second ends of the connecting member; and
c) at least one blocker, the first bone anchor being located between the blocker and the spacer, the blocker located near the sleeve, the blocker being fixed to the tensioned cord.

21. The improvement of claim 20 wherein the concave surface and the convex surface have substantially the same radius.

22. The improvement of claim 20 wherein the blocker is adjacent the first bone anchor.

23. The improvement of claim 20 wherein the blocker engages the sleeve.

24. The improvement of claim 20 further comprising a bumper located between the blocker and the first bone anchor.

25. The improvement of claim 20 wherein the at least one sleeve is a first sleeve and further comprising a second sleeve, the second sleeve being integral with a hard rod.

26. The improvement of claim 25 wherein the second sleeve is fixed to the cord.

27. The improvement of claim 26 wherein the second sleeve has a bore and cooperating pin, the pin extending through the bore and fixing the cord to the second sleeve.

28. The improvement of claim 20 wherein the body portion has an aperture located adjacent the concave surface, the aperture sized for receiving a bone screw shank driving tool through the body portion and into a drive feature of at least one of the first and second bone anchors.
